(19) United States
(12) Patent Application Publication
(10) Pub. No.: US 2025/0101009 A1
(43) Pub. Date: Mar. 27, 2025

(54) Compounds, Compositions and Methods for Treatment of Age-Related Ocular Disorders

(71) Applicants: IVIEW THERAPEUTICS, INC., Cranbury, NJ (US); IVIEW THERAPEUTICS (ZHUHAI) CO., LTD., Zhuhai (CN)

(72) Inventors: Bo Liang, Plainsboro, NJ (US); Guoxin Du, Zhuhai (CN)

(21) Appl. No.: 18/573,860

(22) PCT Filed: Nov. 20, 2023
(86) PCT No.: PCT/US2023/080439

§ 371 Date: Dec. 22, 2023

Related U.S. Application Data
(60) Provisional application No. 63/426,722, filed on Nov. 19, 2022.

(51) Int. Cl. C07D 409/14; A61K 31/4178; A61P 27/02

(52) U.S. Cl. CPC C07D 409/14; A61K 31/4178; A61P 27/02

(57) ABSTRACT

The present invention provides novel bifunctional compounds of Formula (I), including a miotic agent and a disulfide bond reducing agent, connected by a bond or a linker. Such compounds may effectively prevent, treat, and/or ameliorate of age-related ocular disorders, such as presbyopia and cataract.

Pilocarpine-L-(B)ₚ (I)

### CROSS-REFERENCE TO RELATED APPLICATION

This application claims the benefit of U.S. Provisional Application No. 63/426,722, filed on Nov. 19, 2022, the contents of which are incorporated herein by reference in their entireties.

### BACKGROUND OF THE INVENTION

Presbyopia is an eye condition characterized by gradual decrease in accommodation of the eye due to the complex degeneration of the ciliary body, vitreous body, and crystalline lens. Typically becomes noticeable around the age of 40. In 2015, it was estimated that 1.8 billion people had presbyopia worldwide, and the number is projected to increase due to ongoing population growth, and is expected to peak at approximately 2.1 billion by 2030. In the United States, nearly half of the adult population is affected by this condition.

Presbyopia manifests through a range of symptoms, including tendency to hold reading material farther away to make the letters clearer, blurred vision at normal reading distance, eyestrain, or headaches after reading or doing close-up work. It stems from a gradual thickening and loss of viscoelasticity of the natural lens. The loss of lens elasticity may be attributed to oxidized protein sulfhydryl groups within lens fiber cells from intraprotein cross-links that, with time, lead to loss of accommodative amplitude (Clinical Ophthalmology, 2020(14): 3439-3450).

Currently, the primary methods for treating presbyopia include optical correction (e.g., wearing reading glasses and contact lenses), surgical interventions (e.g., corneal laser surgery, refractive lens replacement and scleral expansion) and pharmacological treatment (e.g., 1.25% pilocarpine).

However, the surgical interventions of presbyopia have not been widely accepted due to several factors, particularly the common drawbacks such as the risk of postoperative complications, astigmatism, and inflammation. As such, the surgical intervention may not be the best treatment option.

While reading glasses is a common approach used for correction of presbyopia, prolonged use of reading glasses can lead to side effects, such as dizziness, inconvenience, and irreversible increase of diopter strength. Therefore, reading glasses may not be the optimal solution for many presbyopia patients either.

Thus, there is a growing demand for effective, easy-to-use, and noninvasive approaches for treating presbyopia that do not interfere patients' daily activities. Compared with the above mentioned methods, use of medication for treatment is convenient to use and affordable, while also eliminating the risk of postoperative complications. Currently, the two primary potential mechanisms for treating presbyopia are though miosis or lens softening.

Miosis is a medical term referring to the constriction of the pupil. Muscarinic receptor agonist directly stimulates cholinergic receptors, thereby acting on a subtype of muscarinic receptor (known as M3) located on the iris sphincter muscle, and then causing the muscle to contract and resulting in miosis. The purpose of miosis is to enhance the depth of focus, which in turn leads to improvement of the eyesight.

Currently, most drugs employ the process of miosis to treat presbyopia. Miosis only partially counteracts the induced accommodative myopia at a distance, often leading to blurred distance vision. Common side effects of miotic drugs may encompass headache, eyebrow pain, eye irritation, eye redness, blurred vision, difficulty seeing in dim light, and heightened sensitivity to light. These factors contribute to the limitations in the usage of miotic drugs.

The most important aging changes that lead to presbyopia occur in the eye's lens. With aging, the changes in the size, shape and hardness of the lens result in reduced elasticity of the lens, leading to the occurrence and progression of presbyopia. Therefore, the current pharmaceutical research on presbyopia focuses on softening the lens of patients with presbyopia, so as to reduce the hardness and enhance the elasticity of the lens, thereby achieving effective treatment of presbyopia. This is a scientifically sound strategy.

Preclinical and clinical research results showed that aging of humans is linked with an increase in the amount of lens insoluble protein. Under the environmental stimulation, such as oxidative stress, more and more crystalline proteins bind to the thiol groups of glutathione and cysteine, and finally form disulfide between glutathione/cysteine and crystalline proteins. The accumulation of disulfide in the intercellular space is difficult to dissolve, and can result in the decrease of lens elasticity.

In view of the analysis above, miotics and lens softeners are two main agents for potential treatment of presbyopia. As of now, the FDA has not approved any lens-softening agents, and has only approved one miotic agent for presbyopia, i.e., VUITY™ (1.25% pilocarpine HCl ophthalmic solution). Therefore, treatments currently available to patients remain limited. Development of novel drugs for treating presbyopia that can simultaneously contract pupil and soften lens merits significant attention.

### BRIEF SUMMARY OF THE INVENTION

The present invention provides bifunctional compounds, which include two functional moieties (e.g., a miotic agent and a disulfide bond reducing agent) that are connected by a bond or a linker. For instance, the present invention provides a compound of Formula (I) (as further described below), or an enantiomer, diastereomer, racemate, or a pharmaceutically acceptable salt or solvate thereof, which possesses dual functions of miotic activity and disulfide bond reducing activity. These compounds may effectively prevent, treat, or ameliorate age-related ocular disorders, such as presbyopia and cataract.

The present invention notably overcomes the uncertainty or unpredictability linked with separate administration of a miotic agent and a disulfide bond reducing agent, by chemically bonding the two entities via a proper bond or a linker. This results in a single, novel and effective bifunctional compound, which can be used in treatment of age-related ocular disorders through the mechanism of both miosis and lens softening. The combined actions of such bifunctional compound yield a higher potency and therapeutic effect, as compared to situations when the two moieties are used individually or separately.

In one aspect, the present invention provides a compound of Formula (I):

Pilocarpine-L-(B)ₚ (I)

or an enantiomer, diastereomer, racemate, or a pharmaceutically acceptable salt or solvate thereof, wherein:
p is 1 or 2;
Pilocarpine is

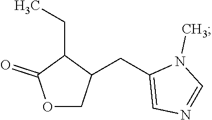

B is

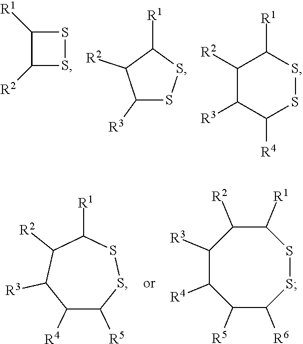

R¹, R², R³, R⁴, R⁵, and R⁶ are each independently H, alkyl, R¹¹—C(O)—O—, R¹¹—C(O)—O-alkylene-, R¹³—O—C(O)—, R¹³—O—C(O)-alkylene-, R¹¹—C(O)—, R¹¹—C(O)-alkylene-, hydroxy, hydroxyalkyl, —NR¹²R^{12′}, cycloalkyl, heterocyclyl, -alkylene-cycloalkyl, -alkylene-heterocyclyl, aryl or heteroaryl, wherein the cycloalkyl, heterocyclyl, -alkylene-cycloalkyl, -alkylene-heterocyclyl, aryl or heteroaryl is optionally substituted with one or more substituents, each substituent optionally and independently selected from alkyl, hydroxyl, hydroxyalkyl, aryl and heterocyclyl;
L (a linker) is a bond, L₁, L₁-L₂, or L₁-L₂-L₃, wherein L₁, L₂, and L₃ are each independently—CH₂—O—, —CH₂—CH₂—O—, —[CH₂—CH₂—O]ₙ₋, —O—CO—, —(CH₃)₂C—O—, —[C₁₋₆ alkylene]CH—O—, —O—, —S—, —CO—, —NH—, —NHCONH—, —C₁₋₆alkylene-, —NH—C₁₋₆alkylene-, —NH—CO—C₁₋₆alkylene-, —C₁₋₆alkylene—NH—CO—, —O—C₁₋₆alkylene-, —CO—O—C₁₋₆alkylene-, —O—CO—C₁₋₆alkylene, —O—C₁₋₆alkylene, —C₁₋₆alkylene-O—CO—C₁₋₆alkylene-, —CH₂—(CH₃)₂CH-phenylene-O—, —C₁₋₆alkylene-phenylene-O—, CH₂—O—PO(—O—)₂, —CH₂—O—PO(OH)—O—, —[C₁₋₆alkylene]CH—O—PO(—O—)₂, —[C₁₋₆alkylene]CH—O—PO(OH)—O—, arylene, cycloalkene, and heterocyclylene, wherein the arylene, cycloalkene or heterocyclylene is optionally substituted with one or more substituents, each substituent optionally and independently selected from halogen, —OH, —SH, —NH₂, —NO₂, C₁₋₄alkyl,—O—C₁₋₄alkyl, or —S—C₁₋₄alkyl;
n is 1, 2, or 3;
each R¹¹ is independently H, OH, C₁₋₆alkyl, optionally substituted cycloalkyl, optionally substituted heterocyclyl, optionally substituted aryl or optionally substituted heteroaryl;
R¹² and R^{12′} are each H or alkyl; and
each R¹³ is independently H, C₁₋₆alkyl, optionally substituted cycloalkyl, optionally substituted heterocyclyl or optionally substituted aryl.

In some embodiments, B is

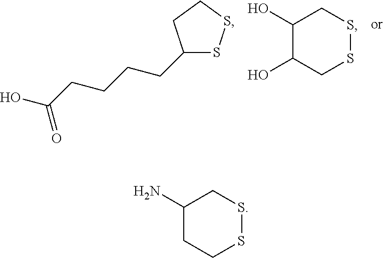

In some further embodiments, B is

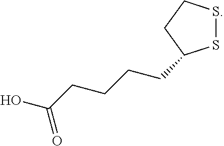 [SMILES: O=C(O)CCCC[C@@H]1CCSS1]

In some embodiments, L is

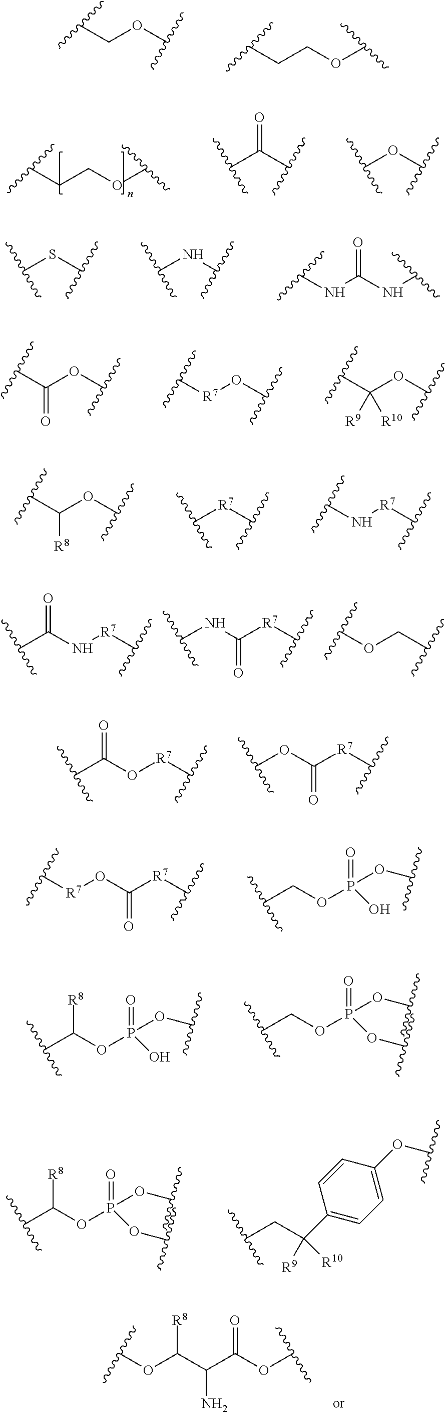

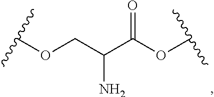

wherein
R⁷ is C₁₋₆ alkylene, C₆₋₁₄ arylene, C₄₋₁₂ cycloalkene, or 4-12-membered heterocyclylene (each being optionally substituted);
R⁸ is C₁₋₆ alkyl, C₆₋₁₄ aryl, C₄₋₁₂ cycloalkyl, 4-12-membered heterocyclyl (each being optionally substituted); and
R⁹ and R¹⁰ are each independently H or C₁₋₆ alkyl.

In some embodiments, the compound is of Formula (II) (including any salt form thereof):

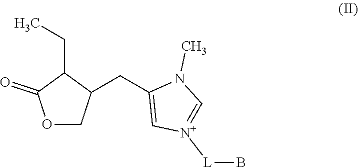

Examples of the compounds of this invention include

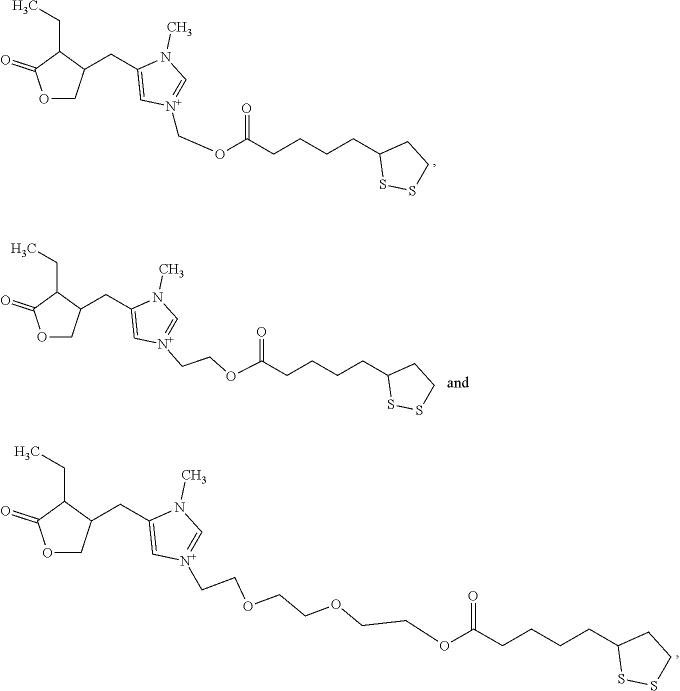 [SMILES: CCC1C(=O)OCC1Cc1c[n+](COC(=O)CCCCC2CCSS2)cn1C ; CCC1C(=O)OCC1Cc1c[n+](CCOC(=O)CCCCC2CCSS2)cn1C ; CCC1C(=O)OCC1Cc1c[n+](CCOCCOCCOC(=O)CCCCC2CCSS2)cn1C]

and salts thereof.

In some embodiments, the compounds of this invention are in the HCl salt form.

In some embodiments, hydrochloride salts of the compounds of this invention include

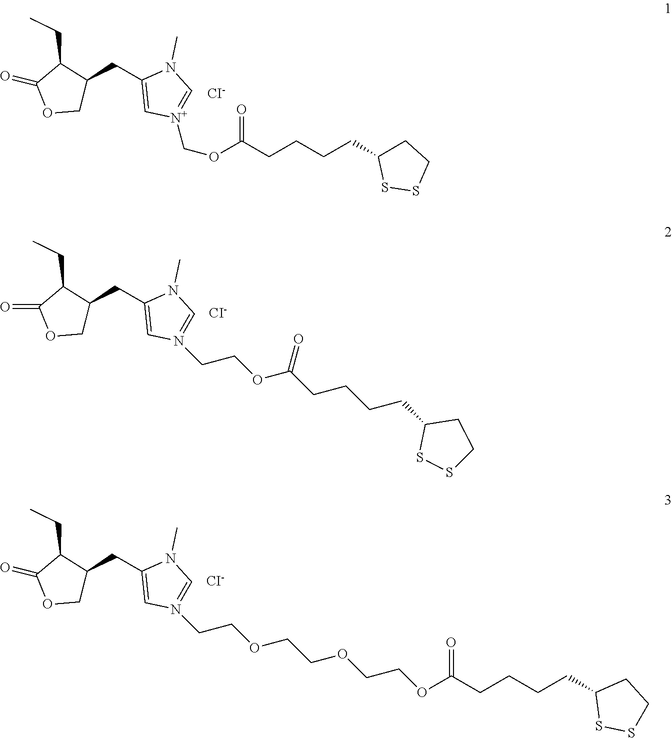

In another aspect, the present invention relates to ophthalmic compositions each including a compound of Formula (I) as defined above, or an enantiomer, diastereomer, racemate, or a pharmaceutically acceptable salt or solvate thereof, and a pharmaceutically acceptable carrier or excipient. Such ophthalmic compositions can be used in prevention, treatment, or management of ophthalmic disease or condition.

In some embodiments, the ophthalmic compositions further include a surfactant, solubilizer, or a biocompatible polysaccharide polymer.

In some embodiments, the ophthalmic compositions further include a gelling agent for increasing the retention time of the bifunctional compounds of this invention on the ocular surface, thereby increasing the bioavailability of the compound of this invention. Examples of the gelling agent include, but are not limited to, sodium carboxymethyl cellulose, sodium hyaluronate, sodium hypromellose, deacetylated gellan gum (DGG), xanthan gum, carrageenan, sodium alginate and a mixture thereof.

In some embodiments, the ophthalmic compositions of this invention further include a pH adjuster, osmotic pressure regulator, or preservative.

In some embodiments, a compound of Formula (I) is contained in the ophthalmic compositions at an amount ranging from about 0.01% to about 5.0% (weight/volume).

In some embodiments, a biocompatible polysaccharide is contained in the ophthalmic compositions at an amount ranging from about 0.1% to about 0.6% (weight/volume).

In some embodiments, the ophthalmic compositions further include a pH adjuster, osmotic pressure adjuster, or preservative.

Yet still another aspect of the present invention provides a method of preventing, treating, or ameliorating an ocular disorder in a subject in need. The method includes administering to the subject in need thereof a therapeutically effective amount of a compound of Formula (I), or an enantiomer, diastereomer, racemate, or a pharmaceutically acceptable salt or solvate thereof, or an ophthalmic composition as described above.

In some embodiments, the ocular disorder is an age-related ocular disorder, such as presbyopia or cataract.

As further described below, the present invention also provides methods for preparing compounds of Formula (I). Moreover, following stability studies, the effectiveness of bifunctional compounds to treat presbyopia were evaluated through in vitro and in vivo efficacy tests to determine the lowest effective concentration.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 illustrates the results of hydrolysis study of Compound 1 with acetylcholinesterase.

FIG. 2 Illustrates the results of hydrolysis study of Compound 2 with acetylcholinesterase.

FIG. 3 illustrates the results of hydrolysis study of Compound 3 with acetylcholinesterase.

FIG. 4 shows the recorded images of rabbit pupil, shrinking under treatment of Vuity™.

FIG. 5 shows the recorded images of rabbit pupil, shrinking under treatment of Compound 1.

FIG. 6 is a graphical representation of the effects of Vuity™ or Compound 1 on rabbit pupil size.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications, and equivalents, which may be included within the spirit and scope of the invention as defined by the claims. Furthermore, in the detailed description of the present invention, numerous specific details are set forth to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

### Definitions

Unless otherwise stated, the following terms used in the specification and claims have the meanings discussed below:

As used herein, the term “or” is meant to include both “and” and “or”. In other words, the term “or” may also be replaced with “and/or”.

As used herein, the term “unsaturated” or “partially unsaturated” refers to a moiety that includes at least one double or triple bond.

As used herein, the term “saturated” refers to a moiety that does not contain a double or triple bond, i.e., the moiety only contains single bonds.

As used herein, the term “alkyl” refers to a saturated straight (i.e., unbranched) or branched hydrocarbon chain radical consisting of carbon and hydrogen atoms, containing no unsaturation, having the stated number of carbon atoms (e.g., C₁₋₁₀ alkyl). Whenever it appears herein, a numerical range such as “1 to 10” refers to each integer in the given range, e.g., “1 to 10 carbon atoms” means that the alkyl group can consist of 1 carbon atom, 2 carbon atoms, 3carbon atoms, 4 carbon atoms, etc., up to and including 10 carbon atoms, although the present definition also covers the occurrence of the term “alkyl” where no numerical range is designated. Examples include, but not limited to, methyl, ethyl, propyl, 2-propyl, n-butyl, iso-butyl, tert-butyl, pentyl, hexyl. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like.

As used herein, the term “substituted alkyl” refers to alkyl substituted with one or more substituents. Examples of the substituent include, but not limited to, halogen, hydroxyl, cyano, amino, alkoxyl, alkoxyalkyl, haloalkyl, alkoxy, amino, methylamino, di-methylamino, sulfone, sulfonamide, aryl, heteroaryl, heterocyclyl, trifluoroethyl, hydroxyethyl, cyanoethyl, methoxyethyl and trifluoropropyl.

The term “alkylene” by itself or as part of another molecule means a divalent radical derived from an alkane, which can be a straight chain or branched chain. In this context, the prefixes (e.g., C₁₋₆) denote the number of carbon atoms, or range of number of carbon atoms. For example, the term “C₁₋₄ alkylene,” as used herein, refers to an alkylene group having from 1 to 4 carbon atoms.

As used herein, the term “alkoxy” or “alkoxyl” refers to a saturated straight or branched hydrocarbon linked to an oxygen atom. Alkoxy group may have the general formula of —O-alkyl. Representative saturated straight chain alkoxys include methoxy, ethoxyl, n-propoxy, n-butoxy, n-pentoxy, n-hextoxy, and the like; while saturated branched alkoxys include isopropoxy, sec-butoxy, isobutoxy, tert-butoxy, isopentoxy, and the like. Cyclic alkoxy are referred to herein as a “cycloalkoxy.” “C₁₋₄ alkoxy” refers to an alkyl with 1, 2, 3, or 4 carbon atoms.

The term “alkoxyalkyl,” as used herein, refers to an alkyl group substituted with one, two, or three alkoxy groups.

As used herein, the term “alkenyl” by itself or as part of another substituent refers to an unsaturated branched or straight-chain having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl, propenyl, and the like.

As used herein, the term “alkynyl” by itself or as part of another substituent refers to carbon chains, which contain at least one carbon-carbon triple bond, and which may be linear or branched or combinations thereof. Examples of alkynyl include ethynyl, propargyl, 3-methyl-1-pentynyl, 2-heptynyl and the like.

As used herein, “alkynylene” refers to a divalent radical derived from an alkynyl, which can be a straight chain or branched chain containing at least one carbon-carbon triple bond. By way of example only, “C₂₋₁₀ alkynylene” indicates that there are two to ten carbon atoms in the alkynylene chain.

As used herein, the term “cycloalkyl” by itself or as part of another substituent refers to a non-aromatic carbon-based ring composed of at least three carbon atoms. The term cycloalkyl includes monocyclic cycloalkyl, bicyclic cycloalkyl, polycyclic cycloalkyl, bridged cycloalkyl, fused cycloalkyl, and spirocycloalkyl groups. In a bridged cycloalkyl, the rings share at least two common non-adjacent atoms. In a fused bicyclic cycloalkyl, two rings share a covalent bond. In a spirocyclic cycloalkyl group, one atom is common to two different rings.

The term “cycloalkene” as used herein refers to a divalent radical derived from the cycloalkyl as defined above, by removal of a hydrogen atom from a ring carbon atom of a cycloalkyl group.

The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted.

The term “heterocycle” or “heterocyclyl” as used herein, refers to a group derived from a monocyclic, bridged bicyclic, fused bicyclic, spirocyclic or polycyclic moiety comprising at least one nonaromatic ring comprising one or more ring-forming heteroatoms independently selected from nitrogen, oxygen, and sulfur. The nitrogen atom may be substituted or unsubstituted (i.e., N or NR wherein R is H or another substituent, if defined). The heterocyclyl can be saturated or partially unsaturated. In certain embodiments, a heterocyclyl may comprises 1 to 4 heteroatoms as ring members. The heterocyclyl groups of the present disclosure can be attached to the parent molecular moiety through a carbon atom or a heteroatom in the group.

The term “heterocyclylene” as used herein, refers to a divalent group derived from an heterocyclyl radical as defined above, by removal of a hydrogen atom from a ring atom of a heterocyclyl group.

As used herein, the term “halo” or “halogen” refers to fluorine (fluoro, -F), chlorine (chloro, -Cl), bromine (bromo, -Br), or iodine (iodo, -I). “Haloalkyl” refers to alkyl as defined above in which one or more of the hydrogen atoms have been replaced with a halogen independently selected from fluoro, chloro, bromo, and iodo. “Fluoroalkyl” means alkyl as defined above wherein one or more hydrogen atoms have been replaced by fluoro atoms. Unless otherwise specified with a number, a haloalkyl can include as many as chemically possible halo atoms as substituents on the alkyl group. For example, fluoroethyl can be -CH2CF3, -CHF-CH3, or -CH2CH2F.

As used herein, the term “hydroxyl” or “hydroxy” refers to the group -OH.

The term “hydroxyalkyl” by itself or as part of another substituent refers to an alkyl group in which one or more of the hydrogen atoms are replaced with a hydroxyl substituent. Thus, the term “hydroxyalkyl” is meant to include monohydroxyalkyl, dihydroxyalkyl, trihydroxyalkyl, etc.

The term “cyano” or “-CN” as used herein refers to a group of -CΞN. The term “cyanoalkyl” as used herein, refers to an alkyl group having at least one -CN substituent.

As used herein, the term “amino” or “amine” as used herein refers to -NH₂.

As used herein, the term “aryl” refers to an all-carbon monocyclic or fused-ring polycyclic (i.e., rings which share adjacent pairs of carbon atoms) groups of 6 to 12 carbon atoms having a completely conjugated pi-electron system. Examples, without limitation, of aryl groups are phenyl, naphthyl and anthracenyl. The “aryl” group can be substituted or unsubstituted.

The term “arylene,” as used herein, refers to a divalent group derived from an aryl radical as defined above, by removal of a hydrogen atom from a ring carbon atom of a aryl group.

As used herein, the term “heteroaryl” refers to a monocyclic or fused ring (i.e., rings which share an adjacent pair of atoms) of 5 to 12 ring atoms containing one, two, three or four ring heteroatoms selected from N, O, or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine, triazole, tetrazole, triazine, carbazole, benzimidazole, benzoxazole, benzothiazole, indazole and quinazoline. The heteroaryl group may be substituted or unsubstituted.

The term “heteroarylene,” as used herein, refers to a divalent group derived from a heteroaryl group, as defined above, by removal of a hydrogen atom from a ring carbon or ring heteroatom of a heteroaryl group.

The above-defined groups may include prefixes and/or suffixes that are commonly used in the art to create additional well-recognized substituent groups. As examples, the term “haloalkoxy” or “haloalkyloxy” refers to a haloalkyl group attached to the parent molecular moiety through an oxygen atom. The term “(haloalkyl)oxyalkyl” refers to an alkyl group substituted with one, two, or three (haloalkyl) oxy groups. As another example, the term “hydroxyalkamino” refers to an amino group substituted with one or two hydroxyalkyl groups.

The term “stereoisomer” refers to isomers of identical constitution that differ only in spatial arrangement of atoms, rather than order of atomic connectivity. When a disclosed compound is named or depicted by structure without indicating stereochemistry, it is understood that the name or structure encompasses all possible stereoisomers, including essentially pure stereoisomers, as well as combination thereof. Enantiomers and diastereomers are examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity.

The term “chiral” refers to the structural characteristic of a molecule that makes it impossible to superimpose it on its mirror image.

As used herein, the term “tautomer” refers to each of two or more isomers of a compound that exist together in equilibrium, and are readily interchanged by migration of an atom or group within the molecule. Thus, this disclosure is intended to cover all possible tautomers even when a structure depicts only one of them.

The term “optional” or “optionally” means that the subsequently described event or circumstance may or may not occur. It is meant to include examples where the event or situation appears and examples where it does not appear. For example, “optionally substituted alkyl” refers to “alkyl” can be substituted, as well as alkyl is unsubstituted.

As used herein, the term “pharmaceutically acceptable” refers to compounds, salts, compositions, dosage forms and other materials which are useful in preparing a pharmaceutical composition that is suitable for veterinary or human pharmaceutical use. The term “pharmaceutically acceptable salt” refers to a salt of a compound that is pharmaceutically acceptable and that possesses (or can be converted to a form that possesses) the desired pharmacological activity of the parent compound.

As used herein, the term “pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

As used herein, the term “therapeutically effective amount” refers to that amount of the compound being administered which will relieve to some extent one or more of the symptoms of the disorder being treated.

As used herein, the term “subject” or “patient” is used interchangeably and as used herein mean any mammal including but not limited to human beings including a human patient or subject to which the compositions of the invention can be administered. The term “mammals” include human patients and non-human primates, as well as experimental animals such as rabbits, rats, and mice, and other animals.

Compounds of the invention may exist as one or more stereoisomers. The various stereoisomers include enantiomers, diastereomers, atropisomers, and geometric isomers. One skilled in the art will appreciate that one stereoisomer may be more active or may exhibit beneficial effects when enriched relative to the other stereoisomer(s) or when separated from the other stereoisomer(s). Additionally, the skilled artisan knows how to separate, enrich or selectively prepare said stereoisomers. Accordingly, the present invention comprises compounds of Formula (I), or an enantiomer, diastereomer, racemate, or a pharmaceutically acceptable salt or solvate thereof. The compounds of the present invention may be present as a mixture of stereoisomers, individual stereoisomers, or as an optically active form.

In addition, a compound of Formula (I) (or salt, prodrug or conjugate thereof) may exhibit polymorphism or may form a solvate with water or an organic solvent. The present invention also encompasses any such polymorphic form, any solvate or any mixture thereof.

### Methods

The following methods are illustrative of select embodiments of the present invention and are not meant to limit the scope of the invention.

Stable quaternary ammonium salts can be produced with tertiary or N-heterocyclic amines. Despite their stability, these quaternary ammonium salts are susceptible to enzymatic hydrolysis by esterase, leading to subsequent spontaneous decomposition. Given the known biological lability of certain quaternary ammonium salts, it is proposed that the novel bifunctional compounds of the present invention are transient inactive derivatives. The proposed mechanism for the release is detailed in reference articles (see, New Water-Soluble Pilocarpine Derivatives with Enhanced and Sustained Muscarinic Activity. Pharmaceutical Research. 1992, 9, 372-377; Kinetics and Mechanism of Hydrolysis of Labile Quaternary Ammonium Derivatives of Tertiary Amines. Journal of Pharmaceutical Sciences. 1982, 71, 729-735), where esterase cleaves the acyl portion of the side chain to form an unstable hydroxylammonium salt or quinone methide which spontaneously liberates the tertiary amine or N-heterocyclic amine. According to this mechanism, the observed miotic activity and disulfide bond reducing activity are interpreted as evidence for the intracellular delivery of the active drug.

Particularly, when pilocarpine is bonded (or connected by a linker) to a disulfide compound, it will increase the lipid solubility of the compound (such as compound of Formula (I)), which will make it easier for the compound to penetrate the cornea and reach the aqueous humor. The compound will be hydrolyzed in aqueous humor to release miotic agent and disulfide bond reducing agent. By doing so, the bioavailability of the compound can be increased, and the dosage of pilocarpine can be reduced.

N-acyloxyalkyl or N-acyloxybenzyl derivatives of tertiary amines or N-heterocyclic amines are stable quaternary ammonium salts in aqueous solutions, which are nonetheless susceptible to enzymatic hydrolysis by esterase and subsequent spontaneous decomposition. Using the two methods mentioned above, tertiary amines or N-heterocyclic amines can be linked to other active moieties to form novel bifunctional compounds.

### Part A—Synthetic Methods

### The Synthetic Route of Compound 1—Scheme 1

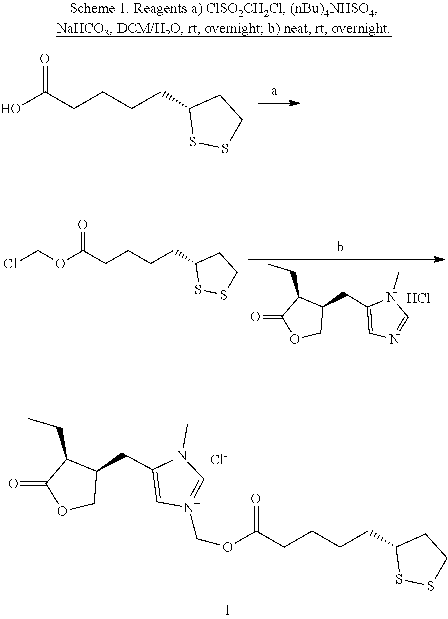

The chloromethyl esters intermediate was obtained by reaction of the corresponding lipoic acid and chloromethyl chlorosulfate under phase transfer conditions. The quaternization reaction occurred on mixing within a range of temperatures between 60 and 90° C.

### The Synthetic Route of Compound 2—Scheme 2

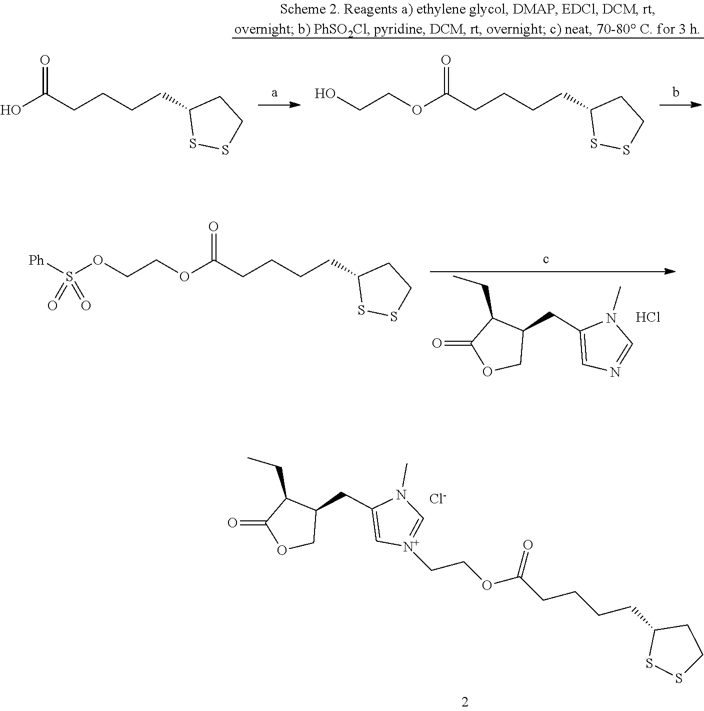

Glycol lipoate was obtained by combining lipoic acid with glycol in acetonitrile. The benzene sulfonyl group then replaces the hydroxyl group and reacts with pilocarpine to obtain the target product.

### The Synthetic Route of Compound 3—Scheme 3

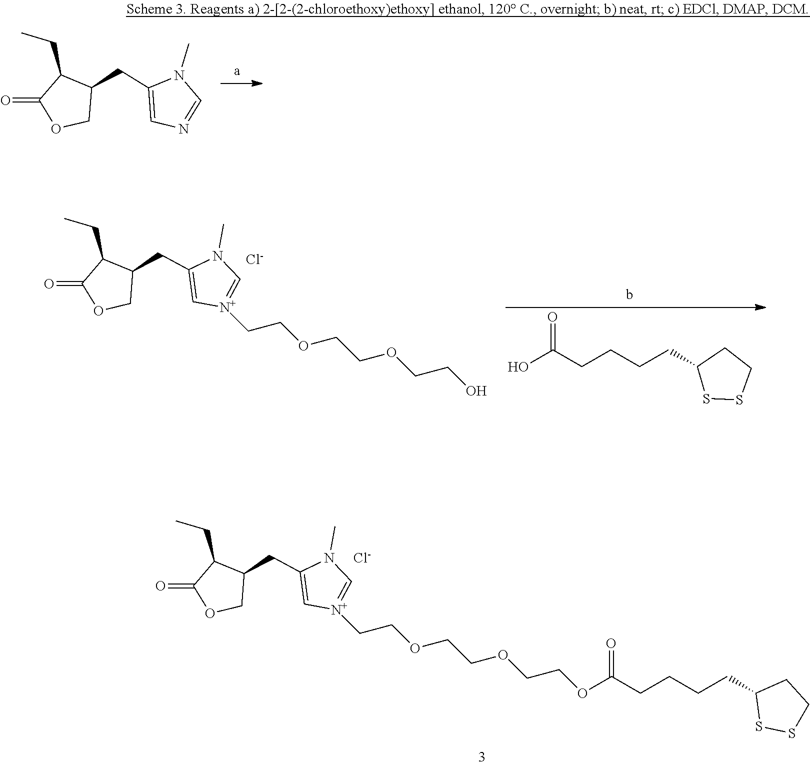 [SMILES: CC[C@@H]1C(=O)OC[C@@H]1Cc1cncn1C ; CC[C@@H]1C(=O)OC[C@@H]1Cc1c[n+](CCOCCOCCO)cn1C ; [Cl-] ; O=C(O)CCCC[C@@H]1CCSS1 ; CC[C@@H]1C(=O)OC[C@@H]1Cc1c[n+](CCOCCOCCOC(=O)CCCC[C@@H]2CCSS2)cn1C ; [Cl-]]

[Pilo-OEG]Cl was obtained from the reaction between oligo-polyethylene glycol chloride and pilocarpine at the imidazole ring. [Pilo-OEG]Cl and lipoic acid are condensed in the presence of EDC and DMAP to obtain ester product.

### Part B—Formulation Study

This present invention provides aqueous formulations each comprising the novel bifunctional compounds (e.g., compounds of Formula (I)), a biocompatible polysaccharide, an osmotic pressure regulator, a pH regulator, a preservative, and water for injection, wherein a gel containing the therapeutic agent is formed in situ upon instillation of the formulation onto eyes. The formulations of the invention are useful for the treatment of presbyopia.

Specifically, the formulations of this invention are aqueous compositions contain novel bifunctional compounds of Formula (I) as the active ingredient and a biocompatible polysaccharide as the in-situ gelling material or matrix.

In some embodiments, the novel bifunctional compounds mentioned above is contained in the aqueous ophthalmic formulation at a concentration ranging from about 0.01% to about 5.0%, from about 0.01% to about 2.5%, from about 0.01% to about 1.0%, from about 0.1% to about 1.0%, or from about 0.1% to about 0.5%, (weight/volume).

As used herein, the term “in situ gel” refers to a system which is applied as a solution or suspension and can undergo rapid sol-to-gel transformation triggered by external stimulus (such as Ion, temperature, pH etc.) on instillation.

The poly saccharide contained in the formulations of this invention may include deacetylated gellan gum (DGG), xanthan, carrageenan, and sodium alginate, or a mixture of these materials.

In some other embodiments, the biocompatible polysaccharide is contained in the aqueous formulation at a concentration ranging from about 0.1% to about 2.0%, from about 0.1% to about 1.0%, from about 0.1% to about 0.6%, from about 0.1% to about 0.3%, from about 0.2% to about 0.5% (weight/volume).

In some other embodiments, the osmotic pressure regulator contained in aqueous ophthalmic formulation includes sodium chloride, glycerol, polyethylene glycol 400 (PEG400), mannitol, or boric acid.

In some other embodiments, the osmotic pressure regulator is contained in the formulation at a concentration ranging from 0.1% to 5.0%, from 0.1% to 4.0%, from 0.1% to 2.5%, from 0.3% to 3.0%, from 0.5% to 2.0%, or from 0.1% to 4.0% (weight/volume). The final osmotic pressure of the formulations may be in the range of 100-500 mOsm, 250-400 mOsm, 250-330 mOsm, or 280-320 mOsm.

The suitable pH regulators in the formulations for this invention include sodium hydroxide, hydrochloride, citric acid/sodium citrate, phosphoric acid/phosphate, acetic acid/sodium acetate, tromethamine or boric acid. The final pH of the formulations may be in the range of 3.0-9.0, 4-8, 5-8, 6-8, 6.8-7.8, or 7.4.

The formulation in the invention may optionally include a preservative. Suitable preservatives may be added to prevent multi-dose package contamination, though the optional antibiotic agent may serve as self-preservative. Such agents may include benzalkonium chloride, benzalkonium bromide, thimerosal, chlorobutanol, methyl paraben, propyl paraben, phenylethyl alcohol, EDTA, sorbic acid, Onamer M, other agents known to those skilled in the art, or a combination thereof. Typically, such preservatives are employed at a level from 0.001% to 1.0% (weight/volume).

### Part C—Hydrolysis Study In Vitro

The commercial esterase was dissolved in simulated tear (pH 7.4). Dissolving an appropriate amount of the compounds of the invention in solvent. The stock solutions were kept in screw-capped vials at 4° C. The reaction was initiated by adding stock solution of the prodrug to preheated esterase solution in a screw-capped vial at 37.5° C. The solution was kept in a water bath at 37.5° C. and at appropriate time intervals. 20 mL aliquot was taken and, after filtration using membrane syringe filter, analyzed immediately by HPLC.

### Part D—Efficacy Evaluation

The ocular activity evaluation based on the miotic effect in rabbits

The pupil size was determined for several compounds in male New Zealand White rabbits. Pilocarpine hydrochloride (Vuity™) was used as positive control. The pupillometer was placed in the eye socket of the rabbit, and the camera was taken 0 min before and 5 min, 10 min, 15 min, 30 min, 45 min and 1 h after dosing. For the latter picture, take three photos at each time point. After the experiment, the pictures were sorted out to observe the pupil change trend after dosing. This method can be used to evaluate whether the miotic agent moiety in the compounds of the invention can constrict the rabbit's pupil.

The invention is further elucidated with specific examples. It is understood that these examples are only used to describe the invention but not to intend to limit the scope of invention. The experimental methods with no specific conditions in the following examples, are usually prepared under conventional conditions in the literature or according to the conditions suggested by the excipient manufacturer. Unless specifically stated, all percentages, ratios, proportions, or fractions in this invention are calculated by weight by weight. Unless specifically defined in this invention, all professional and scientific terms used herein have the same meaning as well-trained personnel may be familiar with. In addition, any methods and materials similar or equivalent to those recorded in this invention can be applied to this invention. The preferred embodiments and materials described herein are used only for exemplary purpose.

### EXAMPLES

### Example 1 Preparation of 3-(((5-((R)-1,2-dithiolan-3-yl)pentanoyl)oxy)methyl)-5-(((3R,4S)-4-ethyl-5-oxotetrahydrofuran-3- yl)methyl)-1-methyl-1H-imidazol-3-ium chloride, compound 1

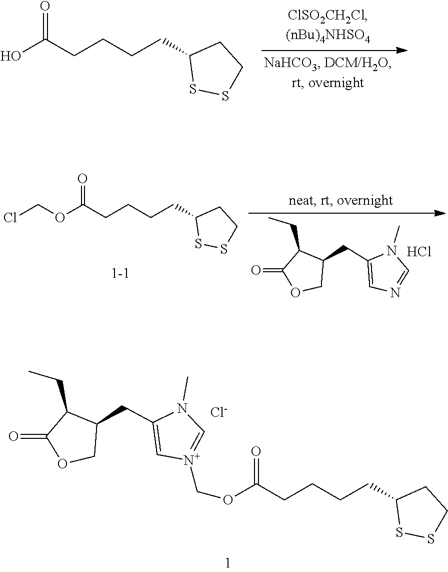

A mixture of pilocarpine (3.0 g, 14.5 mmol), chloromethyl chlorosulfate (2.03 mL), tetrabutylammonium hydrogen sulfate (488 mg, 1.44 mmol), NaHCO3 (4.79 g), DCM (75 mL) and water (75 mL) was stirred vigorously at rt overnight. DCM phase was washed with sat. NaHCO₃, dried over Na₂SO₄, filtered, and concentrated in vacuum. The intermediate 1-1 (2.3 g) was used directly in the next reaction right away.

A mixture of pilocarpine (1.66 g, 8.00 mmol) and intermediate 1-1 (2.13 g, 8.36 mmol) was stirred at rt. The mixture turned sticky and couldn't be stirred in a few minutes. The mixture was stood at rt overnight. The product was obtained by silica-gel chromatography purification (DCM/CH₃OH, 100:0 to 9:1) to give compound 1 as a sticky oil (1.20 g, 32%).

¹H NMR (400 MHz, Chloroform-d) δ 10.83 (s, 1H), 7.48 (s, 1H), 6.31 (d, J=1.7 Hz, 2H), 4.30 (dd, J=9.7, 5.3 Hz, 1H), 4.06 (d, J=2.3 Hz, 1H), 4.03 (s, 3H), 3.59−3.50 (m, 1H), 3.21−3.08 (m, 2H), 2.80−2.67 (m, 2H), 2.61−2.45 (m, 2H), 2.42 (d, J=7.3 Hz, 2H), 1.94−1.86 (m, 2H), 1.74−1.60 (m, 4H), 1.60−1.46 (m, 2H), 1.45−1.35 (m, 2H), 1.12 (t, J=7.4 Hz, 3H).

MS (M-Cl-) m/z: 427.

### Example 2 Preparation of 3-(2-((5-((R)-1,2-dithiolan-3-yl)pentanoyl)oxy)ethyl)-5-(((3R,4S)-4-ethyl-5-oxotetrahydrofuran-3-yl)methyl)-1-methyl-1H-imidazol-3-ium chloride, compound 2

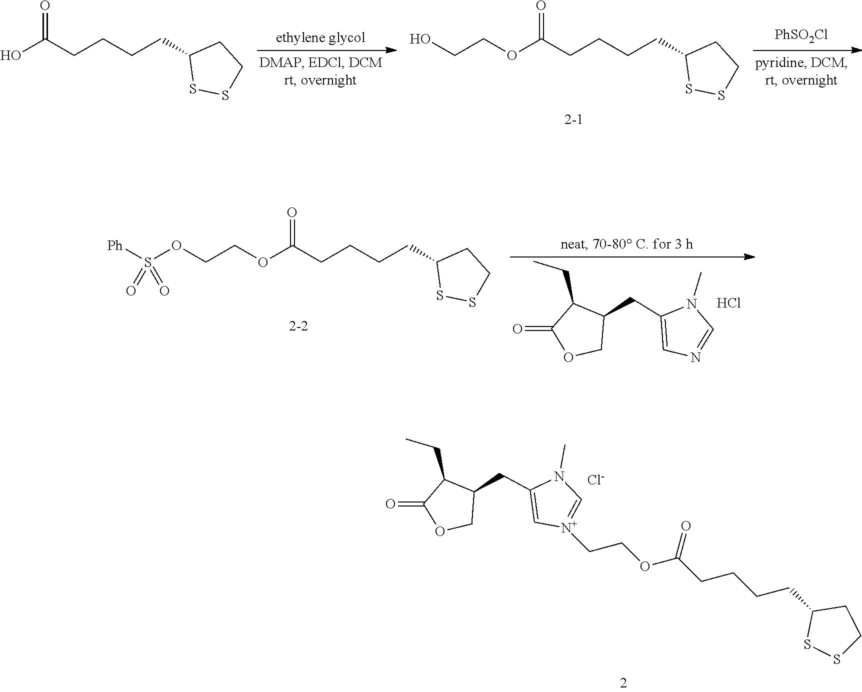

To a mixture of lipoic acid (2.06 g, 10.0 mmol), ethylene glycol (3.30 mL, 60.0 mmol), DMAP (595 mg, 4.85 mmol) in DCM (33 mL) at 0° C. was added EDCI (2.24 g, 11.7 mmol). The mixture was allowed to warm to rt and stirred overnight. Cold water (15 mL) was added and the mixture was stirred for 20 min. The DCM phase was washed with sat NaHCO₃, 0.5N HCl and brine, and then dried over Na₂SO₄, filtered and concentrated in vacuum. The intermediate 2−1 (2.36 g) was used directly in the next reaction.

To a solution of intermediate 2-1 (2.36 g, 9.43 mmol) in DCM (50 mL) at 0° C. was added pyridine (1.50 mL, 18.9 mmol) and phenyl sulfonyl chloride (1.32 mL, 10.4 mmol). The mixture was allowed to warm to rt and stirred at rt overnight. Saturated NaHCO₃ solution was added and the mixture was stirred vigorously until excess phenyl sulfonyl chloride was consumed. The two phases were separated, and DCM phase was washed with 1N HCl and brine, and then dried over Na₂SO₄, filtered and concentrated in vacuum. The product was obtained by silica-gel chromatography purification (Hexanes/EtOAc, 4:1) to give intermediate 2-2 as a sticky oil (2.0 g). The product was dissolved in DCM and used in the next reaction.

To a solution of intermediate 2-2 (2.0 g, 7.85 mmol) in DCM (30 mL) was added pilocarpine (1.50 g, 7.20 mmol). DCM was removed in vacuum and the residue was stirred at 70-80° C. for 3 h. After cooling, the mixture was treated with brine (20 mL) and stirred for 20 min. The two layers were separated and The DCM layer was repeatedly washed with brine (2×) to swap PhSO₃ to Cl. The organic phase was dried over Na₂SO₄, filtered and concentrated in vacuum. The product was obtained by silica-gel chromatography purification (DCM/MeOH, 10:1 to 10:2) to give compound 2 (1.42 g) as a sticky oil.

¹H NMR (500 MHZ, Chloroform-d) δ 10.68 (s, 1H), 7.47 (s, 1H), 4.74−4.64 (m, 2H), 4.51 (t, J=5.0 Hz, 2H), 4.30 (dd, J=9.7, 5.4 Hz, 1H), 4.05 (dd, J=9.8, 2.4 Hz, 1H), 4.00 (s, 3H), 3.59−3.53 (m, 1H), 3.19 (ddd, J=11.0, 7.1, 5.3 Hz, 1H), 3.12 (dt, J=11.1, 6.9 Hz, 1H), 2.79 (dd, J=16.0, 4.1 Hz, 1H), 2.72 (q, J=7.2 Hz, 1H), 2.58 (dd, J=15.9, 11.7 Hz, 1H), 2.51−2.43 (m, 1H), 2.39 (t, J=7.3 Hz, 2H), 1.97−1.85 (m, 2H), 1.77−1.49 (m, 6H), 1.48−1.38 (m, 2H), 1.13 (t, J=7.4 Hz, 3H).

ESI-MS (M-CI-) m/z: 441.

### Example 3

### Preparation of 3-(2-(2-(2-((5-((R)-1,2-dithiolan-3-yl)pentanoyl)oxy)ethoxy)ethoxy)ethyl)-5-(((3R,4S)-4-ethyl- 5-oxotetrahydrofuran-3-yl)methyl)-1-methyl-1H-imidazol-3-ium, compound 3

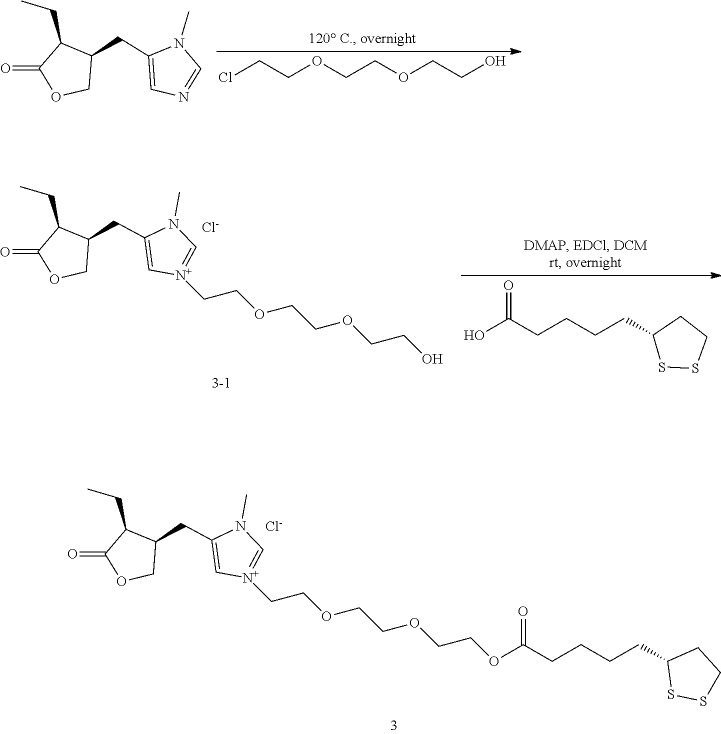

A mixture of Pilocarpine (3.39 g, 16.3 mmol) and 2-[2-(2-chloroethoxy)ethoxy] ethanol (3.02 g, 17.93 mmol) was stirred and heated at 120° C. overnight. The intermediate 3-1 was used in next reaction without further purification. 1H NMR showed it is consistent with the published result.

To a solution of intermediate 3-1 (845 mg, 2.24 mmol), lipoic acid (508 mg, 2.46 mmol) and DMAP (137 mg, 1.12 mmol) in DCM (14 mL) at 0° C. was added EDCI (518 mg, 2.70 mmol). The reaction was allowed to warm to rt and stirred overnight. Cold water (15 mL) was added, and the mixture was stirred for 20 min. The mixture was extracted with DCM (3×20 mL). The combined extracts were washed with brine twice to convert the product to a chloride salt, and then were dried over Na₂SO₄, filtered and concentrated in vacuum. The residue was purified by silica gel column chromatography to give title compound 3 (1.10 g, 87%).

¹H NMR (500 MHz, Chloroform-d) δ 10.51 (s, 1H), 7.52 (s, 1H), 4.50 (t, J=4.5 Hz, 2H), 4.23 (dd, J=9.7, 5.2 Hz, 1H), 4.16 (td, J =4.5, 1.6 Hz, 2H), 3.98 (dd, J =9.7, 2.1 Hz, 1H), 3.91 (s, 3H), 3.82 (t, J=4.6 Hz, 2H), 3.63−3.57 (m, 6H), 3.50 (dq, J=8.9, 6.3 Hz, 1H), 3.15−3.01 (m, 2H), 2.71 (dd, J=15.8, 3.7 Hz, 1H), 2.68−2.62 (m, 1H), 2.50 (dd, J=15.9, 11.7 Hz, 1H), 2.44−2.36 (m, 1H), 2.27 (t, J=7.4 Hz, 2H), 1.87−1.83 (m, 2H), 1.66−1.57 (m, 4H), 1.53−1.43 (m, 2H), 1.42−1.34 (m, 2H), 1.06 (t, J=7.4 Hz, 3H).

ESI-MS (M-CI-) m/z: 529.

### Example 4 Several Compositions Were Prepared With the Compound 1 and Ingredients as Set Forth Below Table 1:

TABLE 1

| Ingredient (% w/v) | Formulation 1 | Formulation 2 | Formulation 3 |
|---|---|---|---|
| Compound 1 | 1.00 | 1.25 | 1.25 |
| Benzalkonium chloride | 0.01 | 0.00 | 0.01 |
| Disodium hydrogen | 0.51 | 0.51 | 0.51 |
| phosphate, anhydrous | | | |
| Sodium dihydrogen | 0.31 | 0.31 | 0.31 |
| phosphate, anhydrous | | | |
| Mannitol | 1.80 | 1.67 | 1.67 |
| Tromethamine | pH 3.0-9.0 | pH 3.0-9.0 | pH 3.0-9.0 |
| Purified water | QS | QS | QS |

### Example 5 Compound 1 Hydrolysis Study with Acetylcholinesterase

The tear fluid was prepared by dissolving sodium chloride (0.67%, w/v), sodium bicarbonate (0.2% w/v) and calcium chloride dihydrate (0.008% w/v) in water and pH was adjusted to 7.4.

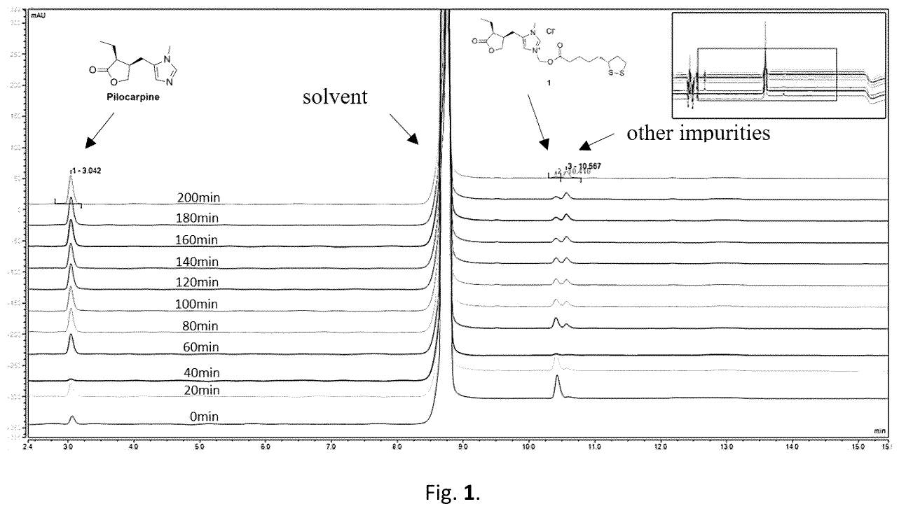

The acetylcholinesterase was dissolved in simulated tear (pH 7.4) to give a concentration of 2.0 mg/ml. Dissolving the appropriate number of compound 1 in pH 7.0 buffer solution to obtain a concentration of 20 mmol/ml. The stock solutions were kept in screw-capped vials at 4° C. The reaction was initiated by adding 20 uL of a stock solution of the compound 1 in pH 7.0 buffer solution to 1 ml of preheated esterase solution in a screw-capped vial at 37.5° C., for a total of 10 identical reaction solutions. The reaction solutions were kept in a water bath at 37.5° C. and at 20 min intervals an aliquot was taken and, after filtration using membrane syringe filter, analyzed immediately by HPLC. The data of enzymatic hydrolysis of compound 1 from 0 min to 200 min were recorded, as shown in Table 2 and FIG. 1.

TABLE 2

| Time | Compound 1 | Pilocarpine | Other impurities |
|---|---|---|---|
| 0 min | 70.29% | 23.30% | 6.41% |
| 20 min | 56.17% | 39.26% | 4.57% |
| 40 min | 34.03% | 44.98% | 20.99% |
| 60 min | 30.13% | 55.97% | 13.90% |
| 80 min | 19.93% | 64.33% | 15.74% |
| 100 min | 17.19% | 65.85% | 16.97% |
| 120 min | 15.06% | 67.67% | 17.27% |
| 140 min | 11.77% | 69.87% | 18.36% |
| 160 min | 8.45% | 72.72% | 18.83% |
| 180 min | 6.94% | 73.80% | 19.26% |
| 200 min | 5.05% | 75.46% | 19.49% |

The data in Table 2 showed that compound 1 was hydrolyzed by acetylcholinesterase almost completely after 200 minutes, and the assay of compound 1 decreased significantly.

### Example 6 Compound 2 Hydrolysis Study With Acetylcholinesterase

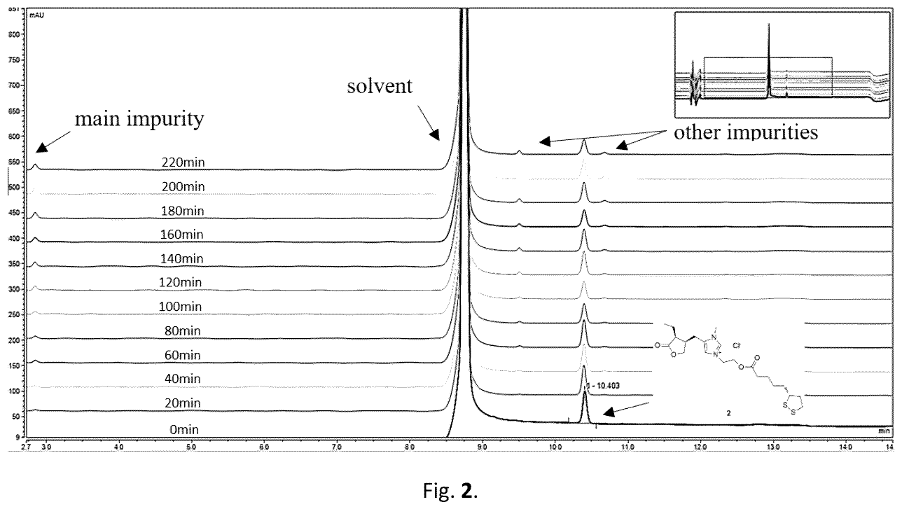

The acetylcholinesterase was dissolved in simulated tear (pH 7.4) to give a concentration of 2.0 mg/ml. Dissolving the appropriate number of compound 2 in pH 7.0 buffer solution to obtain a concentration of 20 mmol/ml. The stock solutions were kept in screw-capped vials at 4° C. The reaction was initiated by adding 20 μL of a stock solution of the compound 2 in pH 7.0 buffer solution to 1 ml of preheated esterase solution in a screw-capped vial at 37.5° C., for a total of 11 identical reaction solutions. The reaction solutions were kept in a water bath at 37.5° C. and at 20 min intervals an aliquot was taken and, after filtration using membrane syringe filter, analyzed immediately by HPLC. The data of enzymatic hydrolysis of compound 2 from 0 min to 220 min were recorded, as shown in Table 3 and FIG. 2.

TABLE 3

| Time | Compound 2 | Main impurity | Other impurities |
|---|---|---|---|
| 0 min | 100.00% | n.a. | n.a. |
| 20 min | 95.69% | 4.31% | n.a. |
| 40 min | 88.08% | 6.36% | 4.63% |
| 60 min | 86.47% | 7.09% | 6.44% |
| 80 min | 80.61% | 10.68% | 8.70% |
| 100 min | 77.56% | 11.94% | 10.51% |
| 120 min | 75.40% | 12.69% | 11.91% |
| 140 min | 72.81% | 13.94% | 13.26% |
| 160 min | 68.28% | 17.08% | 14.64% |
| 180 min | 65.90% | 18.48% | 15.62% |
| 200 min | 63.68% | 19.73% | 16.60% |
| 220 min | 60.88% | 20.63% | 18.49% |

The data in Table 3 showed that compound 2 was hydrolyzed by acetylcholinesterase, and the assay of compound 2 decreased more with time increased.

### Example 7 Compound 3 Hydrolysis Study With Acetylcholinesterase

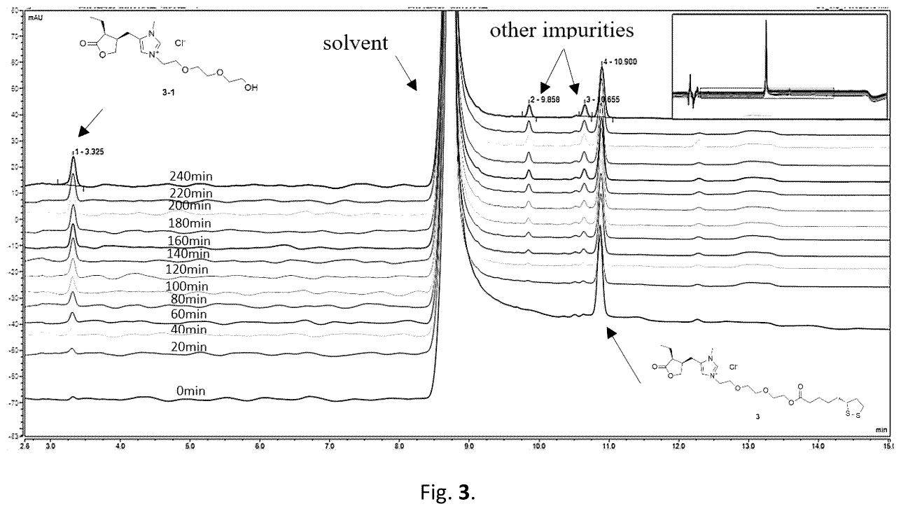

The acetylcholinesterase was dissolved in simulated tear (pH 7.4) to give a concentration of 2.0 mg/ml. Dissolving the appropriate number of compound 3 in pH 7.0 buffer solution to obtain a concentration of 20 mmol/ml. The stock solutions were kept in screw-capped vials at 4° C. The reaction was initiated by adding 20 μL of a stock solution of the compound 3 in pH 7.0 buffer solution to 1 mL of preheated esterase solution in a screw-capped vial at 37.5° C., for a total of 12 identical reaction solutions. The reaction solutions were kept in a water bath at 37.5° C. and at 20 min intervals an aliquot was taken and, after filtration using membrane syringe filter, analyzed immediately by HPLC. The data of enzymatic hydrolysis of compound 3 from 0 min to 240 min were recorded, as shown in Table 4 and FIG. 3.

TABLE 4

| Time | Compound 3 | Intermediate 3-1 | Other impurities |
|---|---|---|---|
| 0 min | 95.40% | n.a. | 3.43% |
| 20 min | 88.83% | 11.17% | 2.83% |
| 40 min | 88.26% | 11.74% | 4.82% |
| 60 min | 79.40% | 13.23% | 8.88% |
| 80 min | 75.30% | 15.77% | 10.33% |
| 100 min | 70.87% | 19.39% | 11.18% |
| 120 min | 68.62% | 20.54% | 12.64% |
| 140 min | 63.48% | 24.15% | 13.85% |
| 160 min | 62.92% | 22.79% | 16.06% |
| 180 min | 59.60% | 24.96% | 17.11% |
| 200 min | 54.50% | 28.47% | 17.51% |
| 220 min | 55.89% | 26.48% | 19.29% |
| 240 min | 51.61% | 30.12% | 19.14% |

The data in Table 4 showed that compound 3 was hydrolyzed by acetylcholinesterase, and the assay of compound 3 decreased more when time increased.

### Example 8 Study on the Miotic Effect of Compound 1 in Rabbits

Under a constant light source (light intensity 40 Lux), the rabbits were fixed in the rabbit box without anesthesia. Before the experiment, the pupil diameter (mm) before drug administration was measured with a pupil measuring ruler and photographed. The eyelid of the rabbit was gently opened, the pipetting gun absorbed the drug drops on the surface of the rabbit cornea (20 μL/eye, binocular administration), and the drug was retained in the conjunctival sac of the rabbit eyelid for 1 min, while the nasolacrimal duct was held down for 1 min. The time of administration was recorded, and the pupil diameter was measured with a pupillometer and photographed with Canon EOS M6 Mark II (SLR) after 5 min, 10 min, 15 min, 30 min, 45 min, 1 h, 2 h, 3 h, 4 h and 5 h, respectively. Each eye was photographed 3 times at each time point. Take the average of three times as the pupil diameter at each time point. The data were expressed using Mean±SEM. Using the GraphPad Prism software, Two-factor ANOVA and Dunnett's multiple comparisons test were used for statistical analysis to compare the differences between groups at different time points. To reduce the interindividual variation of miotic responses which is observed within the sample population when the absolute pupil diameter is measured, the results were reported in terms of miotic activity, which is the percentage reduction in pupil size observed at time t by comparison with time 0.

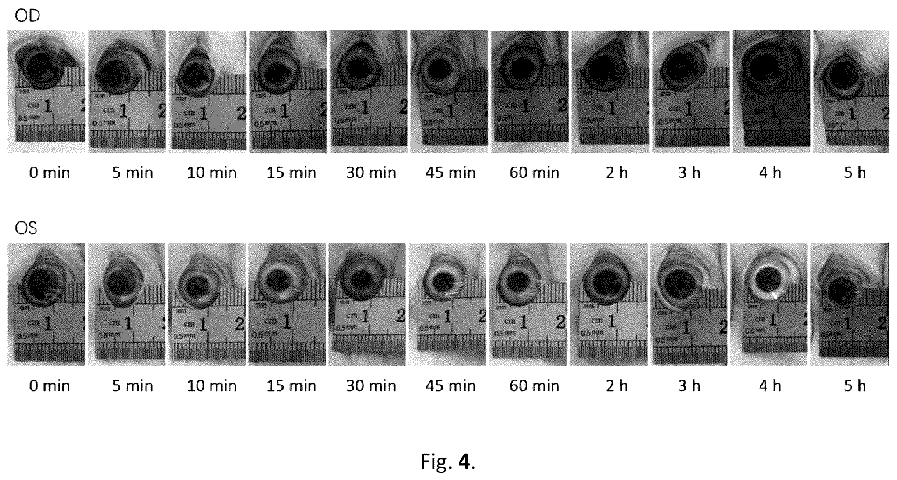

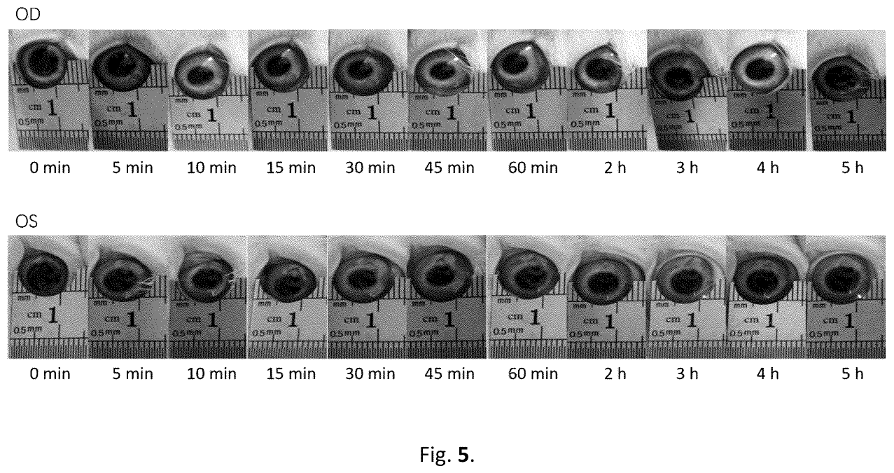

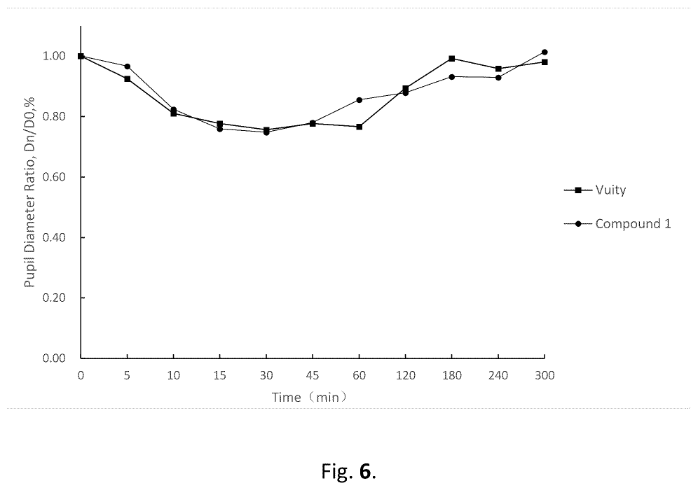

As shown in FIGS. 4, 5, and 6, compound 1 (1.25%) was comparable to the positive control drug (Vuity™M, 1.25%) in terms of miotic effect in rabbits.

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example of a generic series of equivalent or similar features.

1. A compound of Formula (I):
Pilocarpine-L-(B)ₚ (I)
wherein:
p is 1 or 2;
Pilocarpine is 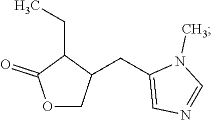
B is 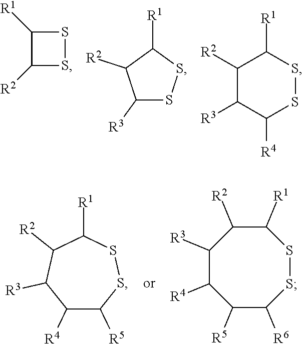
R¹, R², R³, R⁴, R⁵, and R⁶ are each independently H, alkyl, R¹¹—C(O)—O—, R¹¹—C(O)—O-alkylene-, R¹³—O—C(O)—, R¹³—O—C(O)-alkylene-, R¹¹—C(O)—, R¹¹—C(O)-alkylene-, hydroxy, hydroxyalkyl, —NR¹²R^{12′}, cycloalkyl, heterocyclyl, -alkylene-cycloalkyl, -alkylene-heterocyclyl, aryl or heteroaryl, wherein the cycloalkyl, heterocyclyl, -alkylene-cycloalkyl, -alkylene-heterocyclyl, aryl or heteroaryl is optionally substituted with one or more substituents, each substituent optionally and independently selected from alkyl, hydroxyl, hydroxyalkyl, aryl and heterocyclyl;
L (a linker) is a bond, L₁, L₁-L₂, or L₁-L₂-L₃, wherein L₁, L₂, and L₃ are each independently -CH₂—O—, —CH₂—CH₂—O—, —[CH₂—CH₂—O]ₙ—, —O—CO—, —(CH₃)₂C—O—, —[C₁₋₆alkylene]CH—O—, —O—, —S—, —CO—, —NH—, —NHCONH—, —C₁₋₆alkylene-, —NH—C₁₋₆alkylene-, —NH—CO—C₁₋₆alkylene-, —C₁₋₆alkylene-NH—CO—, —O—C₁₋₆alkylene-, —CO—O—C₁₋₆alkylene-, —O—CO—C₁₋₆alkylene, —O—C₁₋₆alkylene, —C₁₋₆alkylene-O—CO—C₁₋₆alkylene-, —CH₂—(CH₃)₂CH-phenylene-O—, —C₁₋₆alkylene-phenylene-O—, —CH₂—O—PO(—O—)₂, —CH₂—O—PO(OH)—O—, —[C₁₋₆alkylene]CH—O—PO(—O—)₂, —[C₁₋₆alkylene]CH—O—PO(OH)—O—, arylene, cycloalkene, and heterocyclylene, wherein the arylene, cycloalkene or heterocyclylene is optionally substituted with one or more substituents, each substitute optionally and independently selected from halogen, —OH, —SH, —NH₂, —NO₂, C₁₋₄alkyl,—O—C₁₋₄alkyl, or —S—C₁₋₄alkyl;
n is 1, 2, or 3;
each R¹¹ is independently H, OH, C₁₋₆alkyl, optionally substituted cycloalkyl, optionally substituted heterocyclyl, optionally substituted aryl or optionally substituted heteroaryl;
R¹² and R^{12′} are each H or alkyl; and
each R¹³ is independently H, C₁₋₆alkyl, optionally substituted cycloalkyl, optionally substituted heterocyclyl or optionally substituted aryl;
or an enantiomer, diastereomer, racemate, or a pharmaceutically acceptable salt or solvate thereof.

2. The compound of claim 1, wherein B is

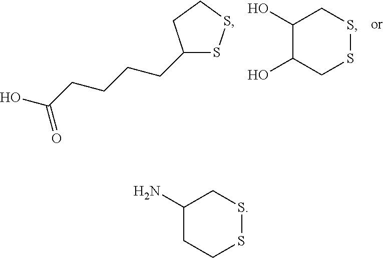

3. The compound of claim 1, wherein B is

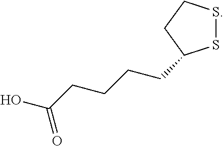 [SMILES: O=C(O)CCCC[C@@H]1CCSS1]

4. The compound of claim 1, wherein L is 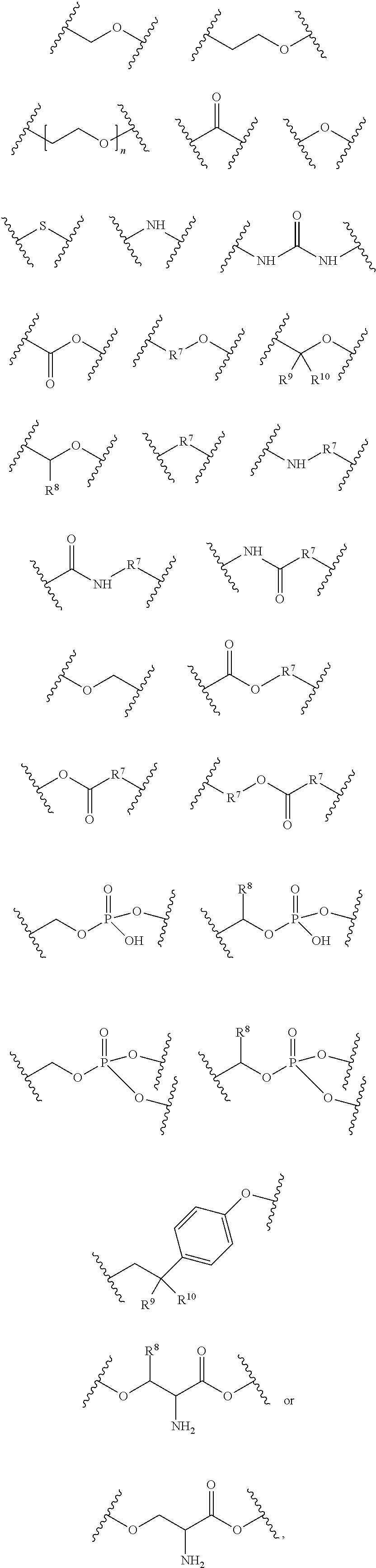
wherein
R⁷ is C₁₋₆ alkylene, optionally substituted C₆₋₁₄ arylene, optionally substituted C₄₋₁₂cycloalkene, or optionally substituted 4-12-membered heterocyclylene;
R⁸ is C₁₋₆ alkyl, optionally substituted C₆₋₁₄ aryl, optionally substituted C₄₋₁₂ cycloalkyl, optionally substituted 4-12-membered heterocyclyl; and
R⁹ and R¹⁰ are each independently H or C₁₋₆ alkyl.

5. The compound of claim 1, wherein the compound is of Formula (II).

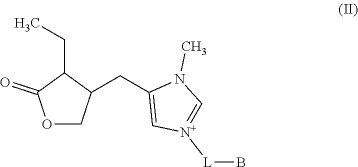

6. The compound of claim 1, wherein the compound is 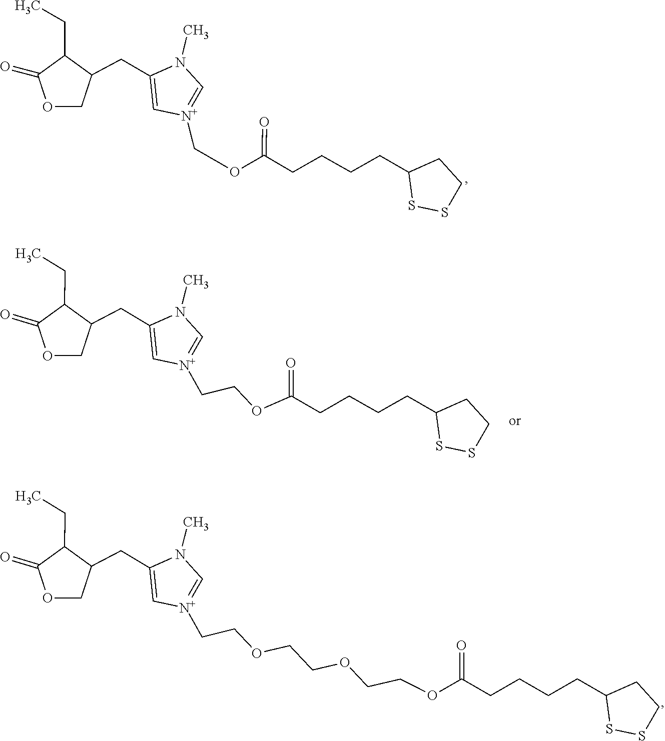 [SMILES: CCC1C(=O)OCC1Cc1c[n+](COC(=O)CCCCC2CCSS2)cn1C ; CCC1C(=O)OCC1Cc1c[n+](CCOC(=O)CCCCC2CCSS2)cn1C ; CCC1C(=O)OCC1Cc1c[n+](CCOCCOCCOC(=O)CCCCC2CCSS2)cn1C]
or a salt thereof.

7. The compound of claim 1, wherein the compound is selected from

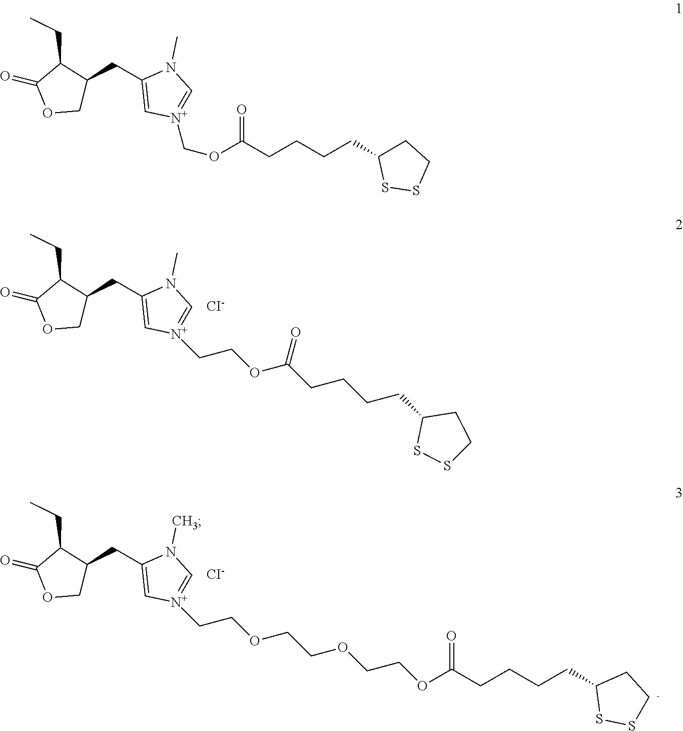

8. An ophthalmic composition comprising a therapeutically effective amount of a compound of claim 1, or an enantiomer, diastereome, racemate, or a pharmaceutically acceptable salt or solvate thereof, and a pharmaceutically acceptable carrier or excipient.

9. The ophthalmic composition of claim 8, further comprising a surfactant, solubilizer, or a biocompatible polysaccharide polymer.

10. The ophthalmic composition of claim 8, further comprising a gelling agent.

11. The ophthalmic composition of claim 10, wherein the gelling agent comprises sodium carboxymethyl cellulose, sodium hyaluronate, sodium hypromellose, deacetylated gellan gum (DGG), xanthan gum, carrageenan, sodium alginate or a mixture thereof.

12. The ophthalmic composition of claim 8, further comprising a pH adjuster, osmotic pressure regulator, or preservative.

13. The ophthalmic composition of claim 8, wherein said compound has an amount ranging from 0.01% to 5.0% (weight/volume) in the composition.

14. The ophthalmic composition of claim 13, wherein the biocompatible polysaccharide has an amount ranging from 0.1% to 0.6% (weight/volume) in the composition.

15. A method of preventing, treating, or ameliorating an ocular disorder in a subject in need, comprising administering to the subject a therapeutically effective amount of a compound of claim 1, or an enantiomer, diastereomer, racemate thereof.

16. The method of claim 15, wherein the ocular disorder is an age-related ocular disorder.

17. The method of claim 16, wherein the age-related ocular disorder comprises presbyopia or cataract.

18. Use of a compound of claim 1 for manufacture of a medicament for treating an ocular disorder.
